(19) United States
(12) Patent Application Publication
(10) Pub. No.: US 2003/0003494 A1
(43) Pub. Date: Jan. 2, 2003

(54) Method for outputting results of comparison of abundances of biopolymers

(76) Inventors: Fumitoshi Ogura, Kanagawa (JP); Takuro Tamura, Kanagawa (JP); Atsumi Tsujimoto, Kanagawa (JP); Azusa Kato, Kanagawa (JP)

Correspondence Address: CHRIS T. MIZUMOTO, Fish & Richardson P.C., Suite 2800, 45 Rockefeller Plaza, New York, NY, 10111, US

(21) Appl. No.: 10/179,065

(22) Filed: Jun. 25, 2002

(30) Foreign Application Priority Data
Jun. 29, 2001 (JP) 198647/2001

(51) Int. Cl. C12Q 1/68; G01N 33/53; G06F 19/00; G01N 33/48; G01N 33/50

(52) U.S. Cl. 435/6; 435/7.1; 702/20

(57) ABSTRACT

For comparatively displaying the abundances of a large number of biopolymer species as measured in two different samples in an efficient and distinguishable manner, the values measured for biopolymer abundances in the two samples are normalized so that the sum totals of the measured values as calculated for both samples become equal to each other and the biopolymer abundance ratios between both samples are displayed in the form of a line graph, with the ordinate (logarithmic scale axis) denoting the abundance ratio for each biopolymer between both samples and the abscissa denoting the biopolymer species. On the same graph, the sum of abundances of each biopolymer in both samples may also be displayed along the Y axis as a second axis.

### BACKGROUND OF THE INVENTION

The present invention relates to a method for outputting the results of analysis of biopolymers in samples with respect to abundances thereof, for example the gene expression levels obtained by gene expression experiments, on a display or printer.

Recent advances in molecular biology experiment techniques have made it possible to determine the abundances of a large number of biopolymers, for example genes or proteins, in one sample or, in other words, determine the gene expression profile or proteome. For storing and utilizing a great mass of information generated by such determination, data processing by means of a computer is essential. In the stage of data analysis for obtaining some or other meaningful piece of information from such data, however, the output of data in the form of graphs or tables from the computer is still subjected in many cases to human evaluation for intuitive judgment. This is probably due to the current situation that the data processing technology using a computer is still inferior to the human empirical information processing ability in finding out an unknown or known law from a mass of information. Nevertheless, by utilizing a computer, it is at least possible to prepare an output on which such outstanding information processing ability of humans can effectively be exercised.

As a technique of displaying the differences in abundances of a large number of biopolymers, such as genes and proteins, between two different samples, there may be mentioned, for example, the Scatchard plot used in gene expression profile presentation (JP-A NO. 342000/1999). Thus, as shown in FIG. 9, one of the X and Y axes is taken for denoting the abundance in one sample (A) and the other for denoting the abundance in the other sample (B), and a spot 901 is plotted for each biopolymer based on the abundances thereof determined in both samples to give a distribution pattern. For each spot, the difference in abundance between the samples A and B can be known from the distance from the straight line (Y=X) 902 having an inclination of 1, and the expression level from the distance from the origin of the coordinates. In an alternative method, the above pattern is drawn on a logarithmic scale with respect to both the X and Y axes, as shown in FIG. 10, so that the distance of a spot from the straight line 1002 having an inclination of 1 may indicate the abundance ratio between the samples A and B. By doing so and assuming that the error of measurement is proportional to the expression level, it becomes possible to ascertain the error of measurement.

In such a comparison, it is a general practice to select certain appropriate control substances for which the assay results can be anticipated and assay these in admixture with the biopolymers in both samples under the same conditions so that the deviations in measured values as occurring between two different samples, namely the deviations in measured values due to the method for preparation of the two samples, the deviations in measured values due to the change in measurement environment when the two samples are assayed in separate or nonparallel experiments, and the deviations in measured values as resulting from the sensitivity and quality of each labeling reagent used in the same or parallel experiments to distinguish the sources of information, can be taken into consideration. And, as shown in FIG. 11, the deviations in measured valued in both samples A and B is determined as the inclination 1103 based on the assay results 1102 for the control substances by the least squares method, for instance, and all the measured values are corrected as indicated by the arrow 1104 so that the above inclination may become 1.

When a large number of biopolymers are involved, it is difficult, in the Scatchard plot technique mentioned above, to indicate which spot represents which biopolymer. In computer displaying, it is possible, by specifying a spot by means of a pointing device, such as a mouse, to indicate the biopolymer represented by the spot. Even in such a case, when the number of spots is great, spot designation becomes difficult due to overlapping of spot sites. In preparing a report about the measurements of abundances of a large number of gene or protein species and the results of data analysis, for instance, there is no means available for effectively indicating where the target genes occur.

For the same reasons, it is also difficult to compare the differences in biopolymer abundance levels as found between two samples with the differences in biopolymer abundance levels as found between other two samples. In many cases of such experiment, it is possible to select one of the two samples as a reference sample to thereby compare the differences among a plurality of different samples through the intermediary of the reference sample treated under the same conditions. In the Scatchard plot, however, there is no means available for indicating which spot is derived from which sample and, in addition, it is difficult to compare spots with one another.

On the other hand, for correcting the deviations in measured values between two samples to be compared with each other, a method is available which comprises preparing controls for which the measured values can be anticipated and carrying out abundance measurements under the same conditions as the measurement target biopolymers; all the measured values for the biopolymers are corrected so that the control abundance values may become equal in both the samples. However, in carrying out measurements in an entrusted analysis service, for instance, the measurements and analysis have to be made in some instances without disclosure of the details of spots for ensuring the confidentiality of the contents of analysis. In such cases, no information can be obtained about the measured values for control spots, which are necessary for correcting the deviations in measured values between two samples, hence the deviations cannot be corrected.

### SUMMARY OF THE INVENTION

In view of the problems discussed above, it is an object of the present invention to provide a method for outputting the results of data analysis in a condition in which a large number of biopolymers are specifiable individually. Another object of the invention is to provide a method for correcting the deviations in measured valued between two samples without using any data for controls.

In accordance with the invention, by which the above problems are to be solved, data normalization is performed using the measured values in each sample so that the deviations in measured valued between the two samples can be corrected. Namely, all the measured values are corrected so that the sum total of the measured values for one sample may become equal to that for the other sample.

Using the corrected values, a graph, for example a line graph, is constructed with the ordinate denoting the abundance ratio for each biopolymer between both samples and the abscissa denoting the biopolymer number. By this, it becomes possible, even in presenting data for a large number of biopolymers, to extend the abscissa, if necessary turn the same back to give a graph having a plurality of tiers, to thereby display the information about the assays of a large number of biopolymers while retaining the ability to supply information about each individual biopolymer. When the ordinate is on the logarithmic scale, the abundance differences can be shown in axial symmetry relative to the line Y=1.0 on the graph. Thus, the extent of deviation in abundance of a certain biopolymer can be judged in a normalized manner in terms of the distance from the line Y=1.0.

According to this method, it is possible, even when there are a plurality of sample pairs, to compare them with one another by drawing graphs on one and the same graph paper while distinguishing them by line color, line species or marking, for instance. The deviation in abundance of each biopolymer between two different samples can be observed in a specific position on the X axis.

In addition, the levels of expression of each biopolymer for both samples are shown on one and the same graph paper. Thus, by superimposing the graphs for both samples, with the ordinate denoting the abundance of each biopolymer and the abscissa denoting the biopolymer species, it becomes possible to show the abundance ratio of each biopolymer in both samples simultaneously with the corresponding absolute abundance values. Where there are a plurality of sample pairs, it is also possible to show mean abundance values for each pair or show mean abundance values for all the samples.

The biopolymer abundance ratios between two samples may be represented by any other method than the line graph method. For example, the data may be presented in the form of a bar graph, each bar extending from the line defined by Y=1.0. It is also possible for the ordinate to denote the biopolymer number and for the abscissa to denote the abundance ratio for each biopolymer between two samples. The graph may be displayed as an image by outputting the data on a display such as a CRT and/or printed on paper or the like medium by feeding the data into a printer.

To sum up, the method for outputting the results of comparison of abundances of biopolymers according to the invention comprises comparing the results obtained by determining the abundances of a plurality of biopolymers 1 to n in two different samples A and B and outputting the results of the comparison and is characterized in that the value obtained by normalization of the ratio Ti/Ci, where Ci is the abundance of a biopolymer i in sample A and Ti is the abundance of the biopolymer in sample B, by means of the mean of the values Tj/Cj (j=1 to n) for the biopolymers 1 to n is outputted as the result of abundance comparison for the biopolymer i.

On that occasion, it is preferred that the results be outputted in the form of a graph with one axis being taken for denoting the biopolymer species and the other for denoting the logarithm of Ti/Ci for each biopolymer. The number of sample pairs is not limited to 1. Thus, it is also possible to output a graph simultaneously showing the Ti/Ci values for a plurality of sample pairs. The graph may be outputted in a divided manner to give a plurality of tiers or sections placed one on the other. Further, it is also possible to output a graph showing the results of abundance comparison for the biopolymer i together with the abundances of the biopolymer i in the two samples.

In accordance with the present invention, it is possible to provide a method for visual representation of the differences in the abundances of various biopolymers as determined in two sample species by which method the deviations in measured values as resulting from assaying of both samples can be normalized and the abundance differences for the respective biopolymers can be represented efficiently and in a distinguishable manner so that the abundance data for the respective biopolymers can be compared between both samples.

Other and further objects, features and advantages of the invention will appear more fully from the following description.

### BRIEF DESCRIPTION OF THE DRAWINGS

In the attached drawings:

FIG. 1 is a schematic representation of a constitution example of the analysis results outputting system according to the invention;

FIG. 2 is a representation of an example of the user interface of the system according to the invention;

FIG. 3 is a schematic representation of the processing flow for outputting (displaying) the results of comparison of biopolymer abundances according to the invention;

FIG. 4 is a representation of specific examples of the biopolymer abundance data obtainable in a gene expression experiment;

FIG. 5 is a flowchart showing an example of the generation of data to be displayed in a biopolymer abundance comparison graph;

FIG. 6 is a schematic representation of expression level data and data normalized for graphic representation;

FIG. 7 is a representation of an output example showing the results of biopolymer abundance comparison;

FIG. 8 is a representation of an example of the case of displaying the results of biopolymer abundance comparison in a plurality of tiers or sections placed one on the other;

FIG. 9 is a graph illustrating the prior art gene expression comparison technique (Scatchard plot);

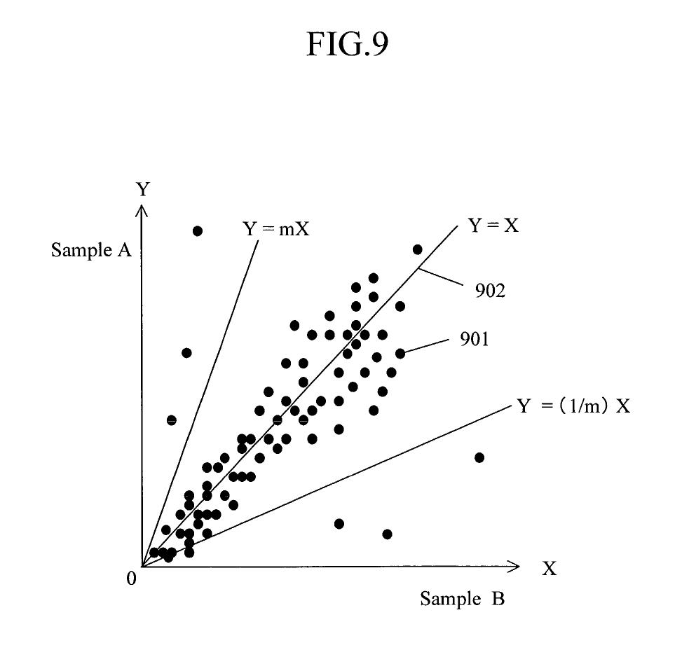

FIG. 10 is a Scatchard plot graph for which logarithmic axes are used; and

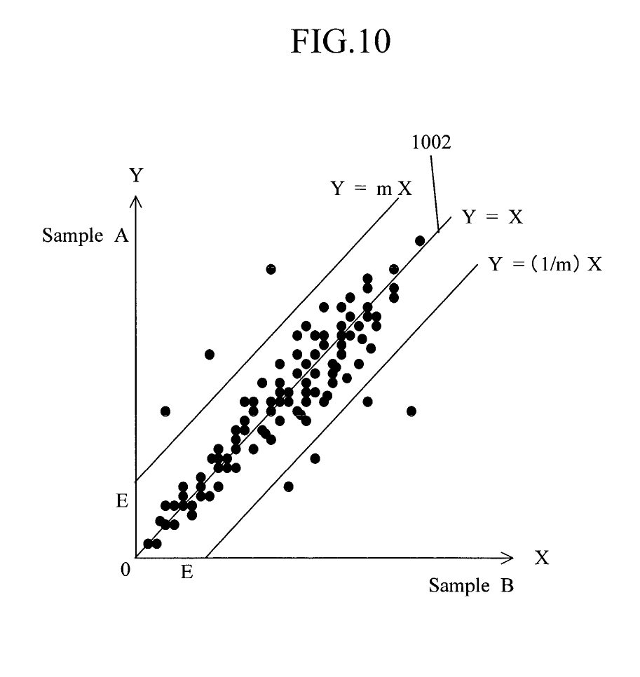

FIG. 11 is a graphic representation of the method for correction of measured values between samples using control substances.

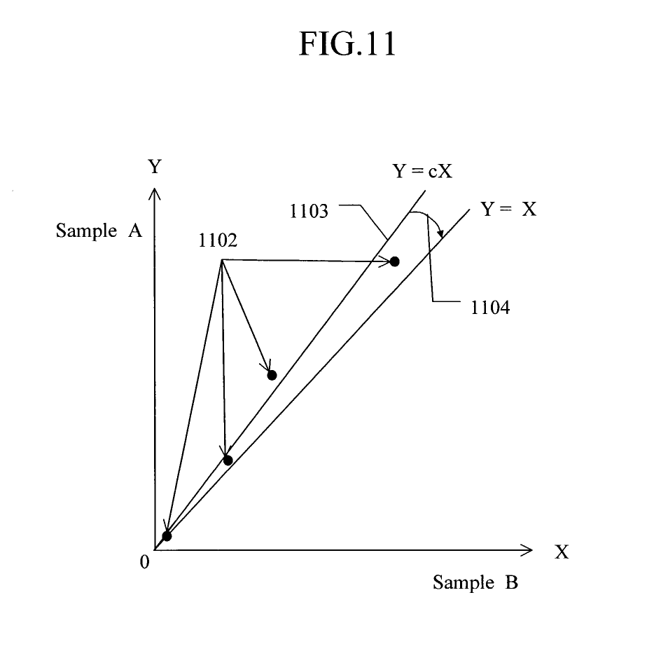

### DETAILED DESCRIPTION OF THE PREFERRED EMBODIMENTS

In the following, some preferred embodiments of the present invention are described referring to the drawings. In the description given herein, the case of outputting (displaying) the results of biopolymer abundance comparison in the form of a line graph is taken as an example.

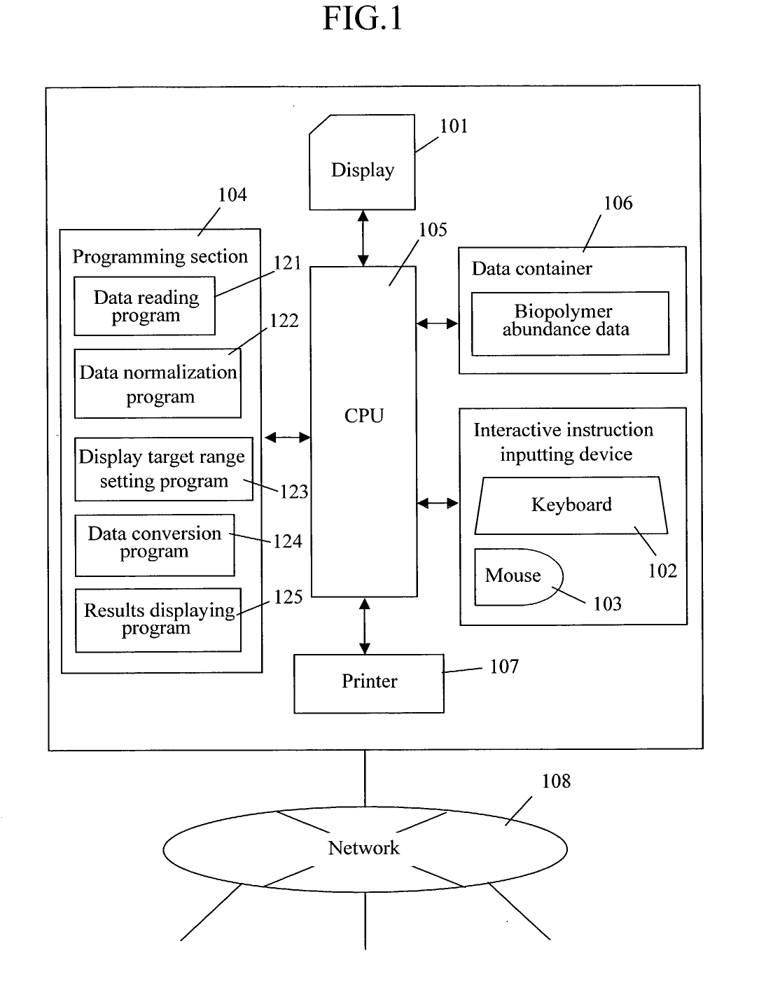

FIG. 1 is a schematic representation of a constitution example of the analysis results outputting system according to the invention. The system according to the invention comprises a data container section 106 containing biopolymer abundance data which are numerical data indicating the extents of abundance of biopolymers occurring in samples, a display 101 for visualizing and displaying the biopolymer abundance data, an input device, such as a keyboard 102 and a mouse 103, intended for inputting values into this system or selecting the values, a programming section 104 containing programs for carrying out calculations for correcting the deviations in measured values between two samples and for carrying out data processing for graphic representation, among others, a CPU 105 which actually performs calculations, and a printer 107 for outputting the results of analysis as displayed in the form of a graph or the like. This system may be connected with a network 108 for data exchange with an external terminal. A data reading program 121, a data normalization program 122, a display target range setting program 123, a data conversion program 124, and a results displaying program 125 are registered and stored in the program section 104 via recording media such as CD-ROMs, DVD-ROMs, MOs and floppy disks, or via a network.

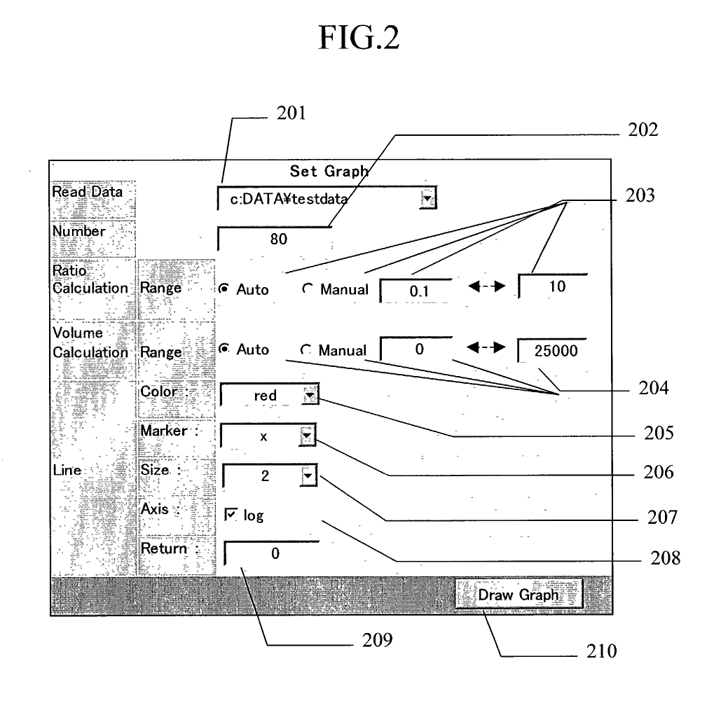

FIG. 2 is a representation of an example of the user interface of the system according to the invention. Various settings are made and graphic representation is carried out using this user interface.

First, in the dialog box 201 for “Read Data”, the address where the data to be read are contained is designated, and the number of biopolymers to be read is inputted in the test box 202 for “Number”. In the input section 203 for “Ratio Calculation”, the range within which the biopolymer abundance values may vary is designated. When “Auto” is selected, the maximum and minimum values calculated from among the target data become the limits and, when “Manual” is selected, the values inputted into the test box serve as the maximum and minimum values. In the input section 204 for “Volume Calculation”, the range within which the biopolymer occurrence index values can vary for both samples is designated. When “Auto” is selected, the maximum and minimum values occurring among the target data become the limits and, when “Manual” is selected, the values inputted into the text box serve as the minimum and maximum values. When “Auto” is selected for “Ratio Calculation” and “Volume Calculation”, all the data are involved in the calculations and the maximum and minimum values are respectively calculated and displayed and editing is also becomes possible. In the input section designated as “Line”, the line graph color (205), marker (206), line species (207), use or nonuse of the logarithmic scale (208), and turning point (209) are set or selected. Upon clicking of the “Draw Graph” button 210, the desired graph is displayed. For selecting a logarithmic axis as the abscissa of the graph, the check box 208 is checked. A plurality of graphs can be displayed in a superimposed manner by repeating the above procedure.

The “turning point” referred to hereinabove is used to select the gene number (starting from gene No. 1 in graphic representation) at which the graph is to be interrupted for continuation in the next tier or section. If, in graphic representation of data for genes Nos. 1 to 100, for instance, 50 is selected as the turning point, a graph for genes Nos. 1 to 50 and a graph for genes Nos. 51 to 100 are displayed in two sections, upper and lower. When 30 is selected as the running point, a graph for genes Nos. 1 to 30, a graph for genes Nos. 31 to 60, a graph for genes Nos. 61 to 90 and a graph for genes Nos. 91 to 100 are displayed in four tiers.

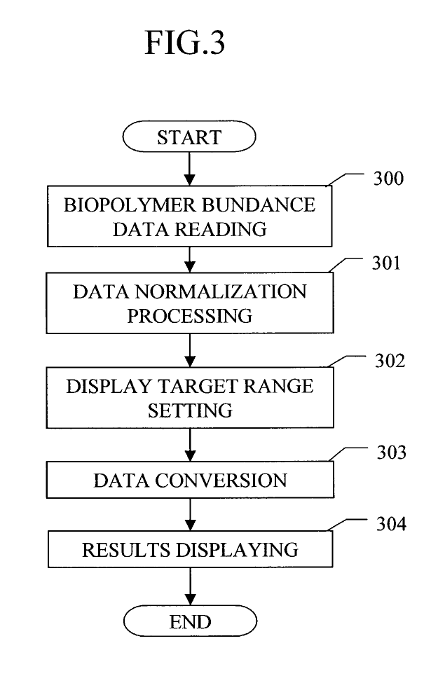

FIG. 3 is a schematic representation of the processing flow for outputting (displaying) the results of comparison of biopolymer abundances according to the invention. The steps to be taken are described below one by one according to this processing flow.

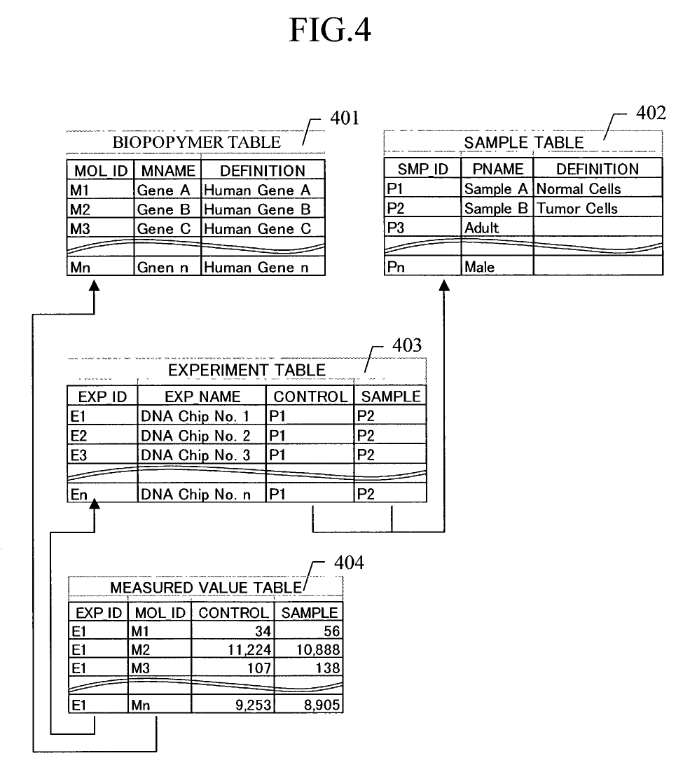

First, the biopolymer abundance data are written into the CPU 105 from the data container 106 (step 300). This processing is performed by the data reading program 121. In FIG. 4, there are shown specific examples of the biopolymer abundance data obtained in a gene expression experiment and suited for processing by the present system. The Sample A and Sample B represented by P1 and P2 under the sample ID (SMP_ID) in the Sample Table (402) are, for example, normal cells A and affected cells B and, in the Biopolymer Table (401), the biopolymers represented by {M1, M2, M3, . . . Mn} under the biopolymer ID (MOL_ID) are, for example, genes which are possibly expressed in cells A and cells B. The values under CONTROL and SAMPLE in the Measured Value Table (404) are relative levels of the genes (abundances of mRNA molecules) in cells A and cells B, respectively, as determined by the experiment. In the Experiment Table (403), the DNA chips used in the experiment and identified by chip number and the samples used as CONTROL and SAMPLE can be checked by E1, E2, . . . under the experiment ID (EXP_ID). By referring to the Experiment Table 403, it is possible to know what kind of experiment has been made.

In a microarray experiment, for instance, nucleic acid sequences complementary to the assay target genes are immobilized at certain positions on a DNA tip and subjected to hybridization reaction with a set of cDNAs derived from cells A and labeled with a fluorescent label R and with a set of cDNAs derived from cells B and labeled with a fluorescence label G. By labeling the cDNA sets derived from different cell species with different fluorescent substances, it becomes possible to measure the relative abundances of cDNAs derived from two cell species in terms of integrated fluorescence intensity values in respective specific wavelength ranges according to the positions P on the DNA chip D in one experiment, namely under the same experimental conditions. For example, in FIG. 4 which shows specific examples of the biopolymer abundance data obtained in a gene expression experiment, the value “34” measured as an integrated fluorescence intensity value in the emission wavelength range of the control sample (P1) labeled with the fluorescent label R and the value “56” measured as an integrated fluorescence intensity value in the emission wavelength range of the target sample (P2) labeled with the fluorescent label G are contained for the biopolymer (M1) on the DNA chip (E1). As for the range of numerical values, the orders of 0 to 65535 (2 bytes) may be taken into consideration in view of the measurement capacity of the currently available fluorescence intensity measurement apparatus (e.g. DNA chip scanner) and the format restriction in storing measurement results as images. It is of course possible to employ a higher order (e.g. 4 bytes, 8 bytes).

Then, normalization processing is carried out for comparative displaying of biopolymer abundances (step 301 in FIG. 3). This processing is performed by the data normalization program 122. If the measured values stored as data as shown in FIG. 4 are plotted as they are on a graph paper, deviations in measured values generally occur between two samples for reasons of sample preparation, experiment, labeling reagent quality and so forth, as mentioned hereinabove. Therefore, in this mode of embodiment of the invention, normalization is effected by normalizing the measurement data derived from each sample so that the total sums of data values for the respective samples become equal to each other, and then calculating the ratios between the two samples.

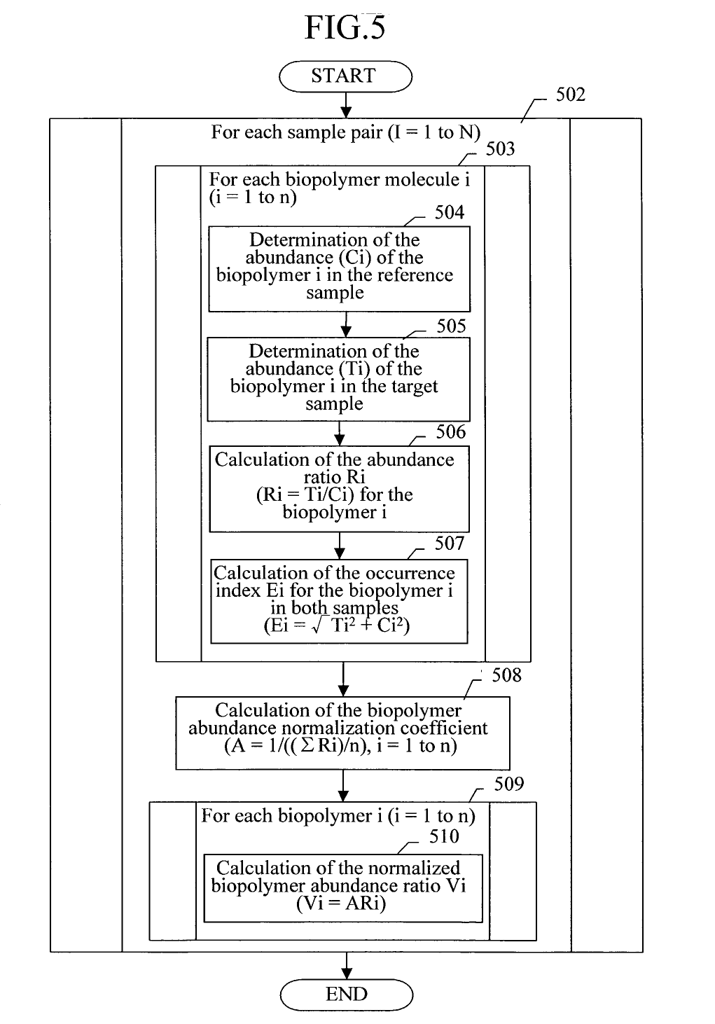

FIG. 5 is a flowchart showing an example of the generation of data to be displayed in a biopolymer abundance comparison graph. An example of the means for data generation for graphic representation in the embodiment of the invention is described below according to this flowchart.

The data generation is carried out for each sample pair (processing 502). In each sample pair, the abundance (Ci) values for the respective biopolymer molecules i (processing 503) are obtained (step 504) from among the biopolymer data for the control sample, which is one of the sample pair parties, and then the abundance data (Ti) are obtained (step 505) from among the biopolymer data for the target sample, which is the other party of the sample pair. Based on the abundance values obtained, the ratios therebetween (Ri=Ti/Ci) are calculated (step 506). Further, for determining the biopolymer occurrence indices Ei in both samples, the Ti and Ci values are plotted on a two-dimensional plane with the X axis denoting one of Ti and Ci and the Y axis denoting the other, and the distances (biopolymer occurrence indices) from the origin are measured (step 507).

The reciprocal of the mean of the biopolymer abundance ratios in the sample pair is calculated, and this is used as a biopolymer abundance normalizing coefficient A (step 508). For all biopolymers in the sample pair (processing 509), the abundance of the biopolymer i is corrected (Vi=ARi) (step 510), and this is used as a normalized biopolymer abundance ratio. For each sample pair, the data necessary for graphic representation can be generated by carrying out the above steps 504 to 510.

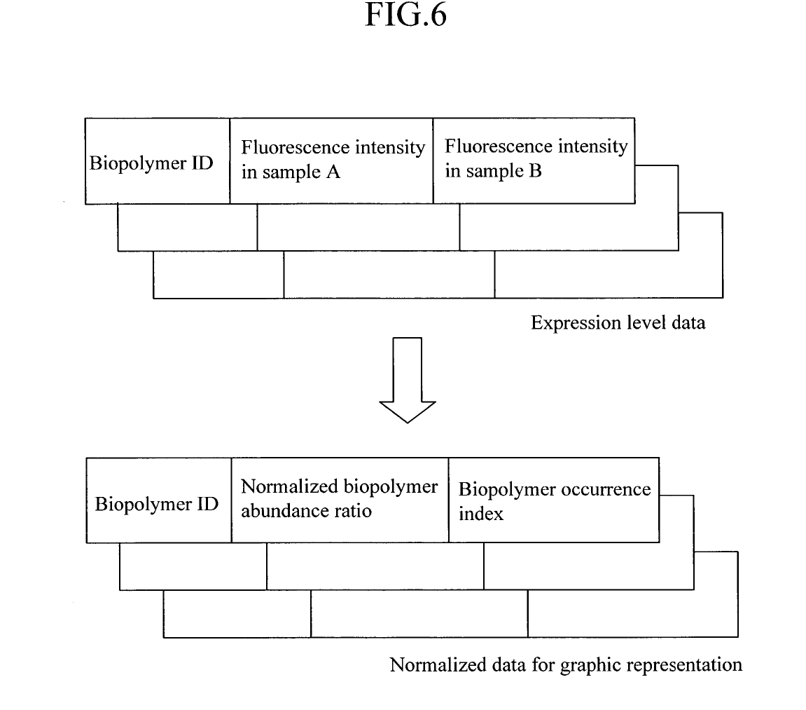

FIG. 6 is a schematic representation of the expression level data contained in the Measurement Value Table 404 and the data normalized for graphic representation by the data normalization processing shown in FIG. 5. The expression level data for each biopolymer are stored in the form of a pair of the fluorescence intensity for sample A and the fluorescence intensity for sample B. When these expression level data are subjected to normalization processing, a pair of the normalized biopolymer abundance ratio data and biopolymer occurrence index data can be obtained for each biopolymer.

Then, the range of data to be employed for graphic representation is selected (step 302 in FIG. 3). This process is carried out by the display target range setting program 123, and the display target range setting is effected by means of the text box 202 of the user interface shown in FIG. 2. When all data after normalization processing in step 301 in FIG. 3 are to be used for graphic representation, the number of the data is inputted in the text box 202. When part of data after normalization processing are to be displayed graphically, the first gene number and last gene number to be used for graphic representation are designated in the text box 202. For graphic representation of the data for gene Nos. 40 to 80, the designation “40-80” is given. When the number of all data are 80, the designation “1-80” may be given for graphic representation of all the data. When only one numerical value is inputted in the text box 202, gene No. 1 to the gene having the number inputted become targets of graphic representation.

Then, the data designated to be included as targets of graphic representation in step 302 shown in FIG. 3 are converted to data for graphic representation (step 303 in FIG. 3). This processing is carried out by means of the data conversion program 124. In this processing, numerical data are converted to dots and line segments for graphic representation. All the data concerned are once read and, within that program, ordinate and abscissa setting is carried out depending on the range of data values. When “Manual” is selected in the input sections 203 and 204 shown in FIG. 2, the respective settings are to be made. A graph is constructed in the program by plotting dots on the thus-prepared graph sheet, and connecting neighboring dots with line segments.

Finally, the results are displayed (outputted) (step 304 in FIG. 3). This processing is carried out by the results displaying program 125.

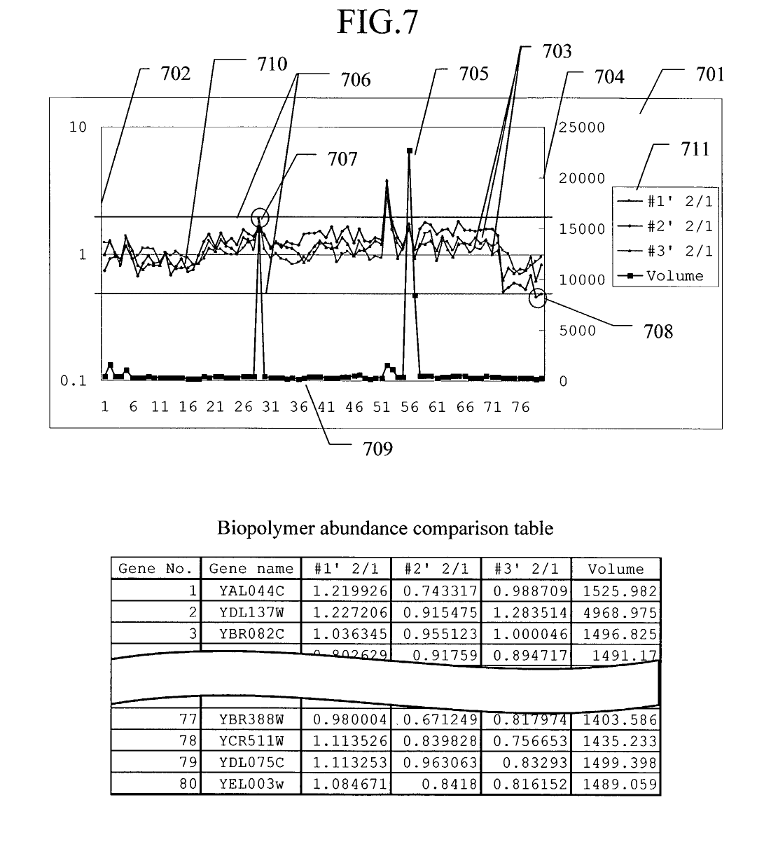

FIG. 7 is a representation of an output example showing the results of biopolymer abundance comparison as displayed (outputted) in step 304 in FIG. 3. In the graph 701 in this example, the biopolymer abundance ratios 703 between two samples, together with the biopolymer occurrence indices 705, are shown for three sample pairs. As for the biopolymer occurrence indices 705, the means of the values for the three pairs are shown in the form of one line graph. The abscissa 709 denotes the biopolymer number (gene No.), while the ordinate is given the scale 702 for normalized biopolymer abundance ratios and the scale 704 for biopolymer occurrence indices. The scale 702 for normalized biopolymer abundance ratios is a logarithmic one. As for the normalized biopolymer abundance ratios, threshold values 706 showing arbitrary ratios (Y=m and Y=1/m) as certain criteria for judging an allowable range of experimental errors are added on both sides of the line 710 (Y=1) (in this example, m=2). Numeral 711 is a legend showing what are represented by the different line graphs.

From the graph 701 shown in FIG. 7, it is evident, for example, that, at point 707, there is a strong deviation in biopolymer abundance ratio and the biopolymer occurrence index shows a high value and, thus, there is a difference between the samples, which is common to the respective sample pairs. At point 708, however, while sharp deviations are found in biopolymer abundance ratio, the biopolymer occurrence index value is low, indicating the possibility of the deviations resulting from experimental errors.

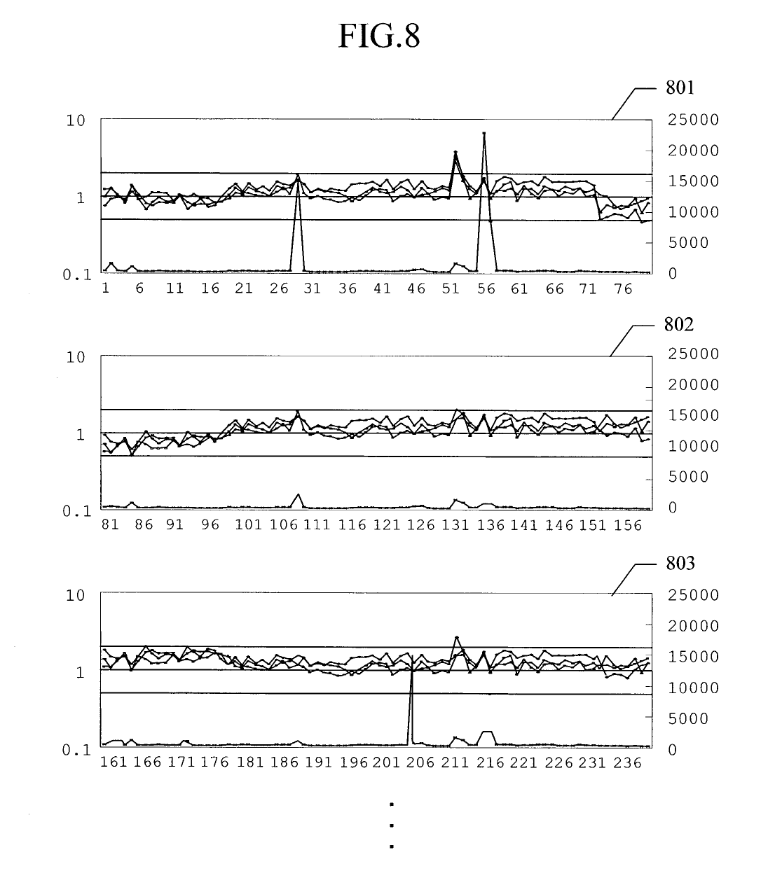

FIG. 8 is a representation of an example of the case of displaying the results of biopolymer abundance comparison in a plurality of tiers or sections. In this example, the biopolymer abundance comparison chart, which is long from side to side, is cut at appropriate points respectively indicating the biopolymer numbers to give segment charts 801, 802 and 803. These segment charts are placed one above the other in the form of a plurality of sections. By this way of presentation, the information concerning a large number of biopolymers can be displayed, or outputted by printing, while distinguishing the individual biopolymers from one another.

As explained hereinabove, the present invention makes it possible, in comparing biopolymer abundance data for two samples, to correct the measured values for comparison thereof between the two samples. It also becomes possible to graphically output data enabling deviation comparison between two different samples as well.

The invention may be embodied in other specific forms without departing from the spirit or essential characteristics thereof. The present embodiment is therefore to be considered in all respects as illustrative and not restrictive, the scope of the invention being indicated by the appended claims rather than by the foregoing description and all changes which come within the meaning and range of equivalency of the claims are therefore intended to be embraced therein.

What is claimed is:

1. A method for outputting the results of comparison of abundances of biopolymers which comprises comparing the results obtained by determining the abundances of a plurality of biopolymers 1 to n in two different samples A and B, and outputting the results of the comparison;
wherein the value obtained by normalization of the ratio Ti/Ci, where Ci is the abundance of a biopolymer i in sample A and Ti is the abundance of the biopolymer in sample B, by means of the mean of the values Tj/Cj (j=1 to n) for the biopolymers 1 to n is outputted as the result of abundance comparison for the biopolymer i.

2. A method for outputting the results of comparison of abundances of biopolymers as claimed in claim 1, wherein the results are outputted in the form of a graph with one axis being taken for denoting the biopolymer species and the other for denoting the logarithm of Ti/Ci for each biopolymer.

3. A method for outputting the results of comparison of abundances of biopolymers as claimed in claim 2, wherein a graph simultaneously showing the Ti/Ci values for a plurality of sample pairs is outputted.

4. A method for outputting the results of comparison of abundances of biopolymers as claimed in claim 2, wherein the graph is outputted in a divided manner to give a plurality of tiers or sections placed one above the other.

5. A method for outputting the results of comparison of abundances of biopolymers as claimed in claim 3, wherein the graph is outputted in a divided manner to give a plurality of tiers or sections placed one above the other.

6. A method for outputting the results of comparison of abundances of biopolymers as claimed in claim 2, wherein a graph showing the results of abundance comparison for the biopolymer i together with the abundances of the biopolymer i in the two samples is outputted.

7. A method for outputting the results of comparison of abundances of biopolymers as claimed in claim 3, wherein a graph showing the results of abundance comparison for the biopolymer i together with the abundances of the biopolymer i in the two samples is outputted.

8. A method for outputting the results of comparison of abundances of biopolymers as claimed in claim 4, wherein a graph showing the results of abundance comparison for the biopolymer i together with the abundances of the biopolymer i in the two samples is outputted.

9. A method for outputting the results of comparison of abundances of biopolymers as claimed in claim 5, wherein a graph showing the results of abundance comparison for the biopolymer i together with the abundances of the biopolymer i in the two samples is outputted.
